(19) United States
(12) Patent Application Publication
(10) Pub. No.: US 2022/0162676 A1
(43) Pub. Date: May 26, 2022

(54) Methods and Kits for Detection of N-4-acetyldeoxycytidine in DNA

(71) Applicant: Active Motif, Inc., Carlsbad, CA (US)

(72) Inventors: Benjamin F. DELATTE, San Diego, CA (US); Mikhail Tchoub, Carlsbad, CA (US); Eddie W. ADAMS, San Diego, CA (US); Joseph M FERNANDEZ, Carlsbad, CA (US)

(21) Appl. No.: 17/616,149

(22) PCT Filed: Dec. 22, 2020
(86) PCT No.: PCT/US2020/066741

§ 371 Date: Dec. 2, 2021

Related U.S. Application Data
(60) Provisional application No. 62/953,062, filed on Dec. 23, 2019.

(51) Int. Cl. C12Q 1/6806; C12Q 1/6869

(52) U.S. Cl. CPC C12Q 1/6806; C12Q 1/6869

(57) ABSTRACT

Provided herein is a new molecular marker in DNA: N-4-acetyldeoxycytidine (“N4-acdC”). Also provided herein are methods of detecting N4-acdC residues in DNA molecules as well as methods of using detected N4-acdC residues, for example in genetic mapping and diagnostics.

### REFERENCE TO RELATED APPLICATIONS

This application claims the benefit of the priority date of U.S. provisional application 62/953,062, filed Dec. 23, 2019, the contents of which are incorporated herein by reference in their entirety.

### BACKGROUND

A method of profiling cytidine acetylation (“N4-acrC”) in RNA has been described in the literature (ACS Chem. Biol. (2017), 12, 2922-2926). The art regarding the analogous RNA modification also includes ACS Chem. Biol. (2018), 140, 12667-12670: A Chemical Signature for Cytidine Acetylation in RNA.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows the chemical structure of N4-acetyldeoxyCytidine.

FIG. 2 shows an exemplary embodiment of N4-acdC DNA immunoprecipitation sequencing.

FIG. 3 shows a control for immunoprecipitation sequencing in which DNA is deacetylated using alkaline reagents such as NH2OH, NaOH, or AMA (NH4OH/MeNH2 mixture). Immunoprecipitation of such material allows one to appreciate the specificity of the N4-acdC antibody used in the assay.

FIG. 4: Genome browser tracks of the ACC-seq assay performed in HeLa cells. Three peaks are visible in the “Mock-IP sample” (gDNA mock-treated then IP'd with a specific N4-acdC antibody). These peaks are not visible in the input track (measure of genomic background such as repetitive regions) nor in the NH2OH-IP track (gDNA NH2OH-treated then IP'd with a specific N4-acdC antibody), showing specificity of the antibody and of the assay.

FIG. 5: Genome browser tracks of the ACC-seq assay performed in HeLa cells. Zoomed-in view of one peak annotated by a red arrow in FIG. 5. As appreciated, signal appears extremely specific with very low background.

FIG. 6: Genome browser tracks of the ACC-seq assay performed in HeLa cells. Zoomed-in view of one peak from the “Mock-IP sample” track. Individual reads are also plotted and show a strand specificity/bias of the modification in genomic DNA (of note, blue and red squares represent strand orientation).

FIG. 7: Genome browser tracks of the ACC-seq assay performed in HeLa cells. Zoomed-in view of one peak from the “Mock-IP sample” track. Individual reads are also plotted and show a strand specificity/bias of the modification in genomic DNA (of note, blue and red squares represent strand orientation).

FIG. 8 compares genetic mapping of G4 structures with N4-acdC residues, indicating significant overlap.

FIGS. 9A-D show genome-wide analyses of the immunoprecipitation sequencing assay performed in HeLa cells. (A-B) show false discovery rate (FDR) as well as FRIP (% of reads inside peaks) scores of the mock-treated and NH2OH-treated samples. In (C), scatter-plots showing the correlation of Mock-treated IP'd tag intensities versus NH2OH-treated IP'd tag intensities. Of note, most of the acdC signal is lost upon chemical deacetylation. In (D), DNA motifs identified by the MEME algorithm.

FIG. 10 shows percent enrichment of Mock-IP sequences vs random sequences: HOMER genomic feature analyses on significant ACC-seq peaks obtained in HeLa cells reveal significant enrichment of acdC (% of IP) in simple repeat regions -red squares- as opposed to what would be expected by chance (% of random).

FIG. 11: Acetylcytidine moiety can be reduced to N4-acetyl-3,4,5,6-tetrahydrocytidine by treatment with a reducing agent. Subsequent removal of acetyl group (via chemical or enzymatic treatment) yields nucleophilic amino group which can be used to attach a reporter group (e.g. biotin, fluorophore) into DNA sequence.

FIG. 12 shows an exemplary embodiment of modified methylase assisted bisulfite/chemical modification-assisted bisulfite sequencing.

FIGS. 13A-B shows an exemplary method of differential digestion-mediated DNA sequencing.

### SUMMARY

It has been discovered that an analogous modification exists in DNA (N4-acetyldeoxyCytidine (“N4-acdC”)). This is the first N4-acetyldeoxycytidine detection and mapping method developed for DNA. The novel ACC-Seq (ACetylCytidine -sequencing) method described below modifies and improves the above-referenced RNA method and adapts it for DNA and next generation sequencing.

In one aspect provided herein is a method of mapping N4-acetyldeoxyCytidine DNA modifications comprising: a) preparing 2 samples of the same DNA; b) removing the epitope recognized by an N4-acetyldeoxyCytidine binder in 1 of said samples, including by treating said sample with a strong nucleophile, such as hydroxylamine or sodium hydroxide; c) subjecting both samples to immunoprecipitation with said N4-acetyldeoxyCytidine binder, such as an antibody, and sequencing; d) comparing the sequence data from step 3, and mapping where peaks are found in the untreated sample but are not present, or reduced in the treated sample.

### DETAILED DESCRIPTION

### I. Introduction

It has been discovered that naturally occurring DNA molecules can comprise N4-acetyldeoxyCytidine (“N4-acdC”) modified nucleotides. Rather than comprising an amino group at the position 4 carbon of cytosine, the amino group is substituted with an acetyl group.

Methods provided herein allow for mapping of N4-acdC residues in DNA molecules. In some embodiments, the methods include enriching a sample for DNA molecules comprising N4-acdC residues. Samples enriched for N4-acdC residues can be mapped to a reference genome to identify the position of the and 4-acdC residues, and analyzed in other ways.

Disclosed herein are methods of enriching, identifying and mapping N4-acetyldeoxyCytidine modified DNA throughout genomes of interest (e.g., bacterial, viral, human). Doing this allows one to determine the specificity of the peaks one observes via sequencing by maintaining a parallel control sample in which all N4-acetyldeoxyCytidine moieties have been removed via chemical deacetylation prior to immunoprecipitation. Therefore, coupling immunoprecipitation of N4-acetyldeoxyCytidine—containing DNA +/− chemical deacetylation gives us a genome-wide view of where this modification is found. This method is referred to as “ACC-Seq” for ‘ACetylCytidine-Sequencing’.

In certain embodiments, DNA is processed to convert N4-acdC into the more stable form, N4-acetyl-3,4,5,6-tetrahydrocytidine (“N4-athC”). All processes described herein can be modified to use this form of DNA. Such methods may have to accommodate appropriate changes, for example the use an antibody or protein that binds N4-acetyl-3,4,5,6-tetrahydrocytidine rather than N4-acdC.

Methods described herein achieve three main things:

1.) They provide methods of detecting a modification in DNA that hitherto remained undetectable and, as a consequence, unknown;

2.) They provide a method of mapping G-quadruplex structures throughout the genome as this modification is highly associated with G-quadruplex structures. G-quadruplex secondary structures (G4) are formed in nucleic acids by sequences that are rich in guanine. They are helical in shape and contain guanine tetrads that can form from one, two or four strands.

3.) They enable the identification and tracking of N4-acetyldeoxyCytidine-associated biomarkers in diagnostic and clinical applications.

Applications include:

1.) Kits for the above for research and development.

2.) Other N4-acdC sequencing/detection kits based on the strategies detailed herein.

3.) Diagnostic assays detecting N4-acdC-containing biomarkers.

4.) Identifying readers of N4-acdC by incubating the acetylated and deacetylated (as a control) consensus sequence with nuclear extracts.

Here, “readers” refers to or includes proteins or protein domains capable of recognizing the N4-acdC structure (whether it has been synthetized in vitro, or pulled-down by ACC-seq) and “nuclear extracts” refers to or includes nuclear proteins prepared from cells or tissues wherein this modification is being examined.

### II. Enriching Nucleic Acids for Sequences Comprising N4-AcdC

Methods provided herein allow for enrichment of nucleic acids, and in particular, DNA, having a modified cytosine residue, N4-acdC. Molecules enriched for N4-acdC can be subject to analysis, including nucleic acid sequencing. Nucleotide sequence reads thus produced can be mapped to a reference genome to identify the location of the modified residues.

### A. Samples Comprising Nucleic Acids

Any nucleic acid molecule comprising N4-acdC residues can be the subject of the methods disclosed herein. This includes both DNA and, in certain embodiments, RNA.

Nucleic acids can be sourced from any biological sample, including, for example, from a virus, a cell or cells or microbiome of any living organism. This includes both prokaryotes (such as archaea and bacteria) and eukaryotes (such as plants, animals and fungi). Animals include, without limitation, insects, fish, amphibians, reptiles, birds and mammals. Mammals include, without limitation, carnivores (e.g., dogs and cats), artiodactyls (e.g., cattle, goats, sheep, pigs), lagomorphs (e.g. rabbits), perissodactyls (e.g., horses), rodents (e.g., mice, rats), and primates (e.g., humans and nonhuman primates (e.g., monkeys, chimpanzees, baboons, gorillas).

Nucleic acids can come from a cell line, a tissue, an organ or a bodily fluid. Cells from any organ or organ system of an animal. Such organs include, without limitation, heart, brain, kidney, liver, lungs, muscle, blood. Body fluids that can be sources of nucleic acids include, without limitation blood, plasma, serum, saliva, sputum, mucus, lymphatic fluid, urine, semen, cerebrospinal fluid or amniotic fluid. Organ systems include, without limitation, muscular system, digestive system, respiratory system, urinary system, reproductive system, endocrine system, circulatory system, nervous system, and integumentary system. A sample can be prepared, for example, by biopsy. This includes both solid tissue biopsy and liquid biopsy. The sample can comprise cell-free DNA (“cfDNA”), such as circulating tumor DNA. Nucleic acid fragments can have a length between about 100 to about 800 nucleotides or 350 to 450 nucleotides, e.g., around 400 nucleotides. cfDNA typically has a size of about 120-220 nucleotides.

Samples comprising nucleic acids can be sourced from a subject having or suspected of having a pathological state. Such states include, without limitation, hyperplasia, hypertrophy, atrophy, and metaplasia, including, e.g., cancer (e.g., a cancer biopsy sample). Other pathologies include neuronal diseases (e.g., Alzheimer's Disease, Amyotrophic Lateral Sclerosis, Creutzfeldt-Jakob Disease, Friedreich's Ataxia, Multiple Sclerosis).

Nucleic acids can be naked nucleic acids, that is, with no proteins attached. Alternatively, nucleic acids can be in the form of chromatin. As used herein, the term “chromatin” refers to a complex of DNA and histone and/or non-histone proteins.

DNA can be purified in the form of chromatin. DNA from chromatin can be enriched by methods such as chromatin immunoprecipitation (ChIP) and transposon-assisted chromatin immunoprecipitation. ChIP methods typically involve crosslinking chromatin in order to covalently bind proteins to nucleic acids. Chromatin can be crosslinked while still in the cell. The chromatin then can be sheared. Nucleic acids having particular proteins bound thereto, such as histones, can be immunoprecipitated using an antibody directed against the target protein. In transposon-assisted chromatin immunoprecipitation, the antibody against the target protein is bound, directly or indirectly, to a transposome. A transposome comprises a transposase attached to a transposon. Upon finding its target, the transposon is inserted into the DNA. When transposons are provided with primer binding sites, nucleic acid positioned between the primer binding sites can be amplified. (See, for example, U.S. Pat. No. 10,689,643, Jelinek et al.)

### B. Nucleotides and Modified Forms Thereof

Nucleotides in RNA and DNA can exist in their native form or in various modified forms. Cytosine can exist in several different forms.

Reference to a nucleotide, in contrast to a base, by letter, can refer to either the “ribo” version or the “deoxyribo” version, unless otherwise specified. In general, nucleotides in DNA will be in the “deoxyribo” version, while nucleotides in RNA will be in the “ribo” form.

The term “modified nucleotide” refers to a derivative of cytosine, adenine, guanine, thymine or uracil. The term “modified cytosine” refers to a derivative of cytosine, typically derivatized with a chemical moiety at position 5 or position 4. The terms “cytosine” and “cytidine” are sometimes uses interchangeably, while “cytidine” can refer to the nucleotide residue in a polynucleotide.

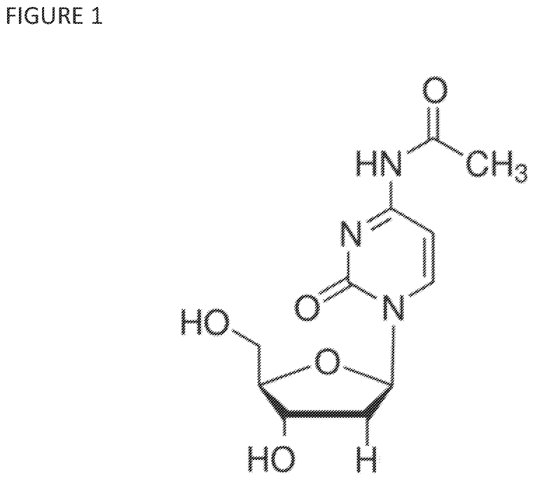

A modified form of cytosine is N-4-acetyldeoxycytidine (“NA-acdC”). The chemical structure for N4-acdC is shown in FIG. 1. In certain methods disclosed herein the acetyl group attached to the nitrogen at the 4-carbon of N4-acdC is converted to an amino group. This process is referred to as “deacetylation”. In other embodiments, the acetyl group attached to the nitrogen at the 4-carbon of N4-acdC is treated with a reducing agent, converting N4-acetyldeoxycytidine (“N4-acdC”) residues in the nucleic acid molecules into N4-acetyl-3,4,5,6-tetrahydrocytidine residues. In other embodiments, deacetylating the N4-acetyl-3,4,5,6-tetrahydrocytidine residues will produce primary nucleophilic amines. N-4-acetyldeoxycytidine can be successfully deacetylated using alkaline reagents such as, but not limited to, NaOH, NH₂OH, and Ammonium Hydroxide/aqueous MethylAmine (“AMA reagent”).

Other modified cytosines include, in increasing order of oxidation state, 5 methylcytosine (“5mC”), 5 hydroxymethylcytosine (“5hmC”), 5 formylcytosine (“5fC”) and 5 carboxylcytosine (“5caC”).

The 4-amino group on cytosine can be converted to a carbonyl group. This process is referred to as “deamination”. In this instance, the base is now uracil. Deamination of cytosine or a modified cytosine by the replacement of the amino group with a carbonyl group at position 4 converts cytosine or a modified cytosine into uracil.

### C. Fragmentation Of Nucleic Acids

In certain embodiments nucleic acids, such as DNA, comprising N4-AcdC are fragmented. Nucleic acids can be fragmented by any methods known in the art including, without limitation, sonication shearing and enzymatic fragmentation, e.g., using endonucleases such as restriction endonucleases. As used herein, the terms “enrichment” and “purification” refer to processes in which molecular species, such as nucleic acids comprising N4-acdC residues, are relatively more numerous (e.g., on a molar basis, more abundant) than other molecular species of the same type, such as nucleic acids in general, in a composition after a step of enrichment or purification.

### D. Isolation Of Nucleic Acids Comprising N4-AcdC Residues by Immunoprecipitation

Compositions comprising nucleic acids can be enriched for molecules comprising N4-acdC residues by specific binding methods. These include, for example, binding with an antibody specific for N4-acdC. Such antibodies can be prepared by standard methods for antibody preparation on the art. Such antibodies also are commercially available, for example, from Abcam (ab252215). (See world wide web site abcam.com/n4-acetylcytidine-ac4c-antibody-eprnci-184-128-ab252215.html).

As used herein, the term “antibody” includes (1) whole immunoglobulins (two light chains and two heavy chains, e.g., a tetramer); (2) an immunoglobulin polypeptide (a light chain or a heavy chain), (3) an antibody fragment, such as Fv (a monovalent or bi-valent variable region fragment, and can encompass only the variable regions (e.g., V_{L} and/or V_{H}), Fab (V_{L}C_{L} V_{H}C_{H}), F(ab′)2, Fv (V_{L}V_{H}), scFv (single chain Fv) (a polypeptide comprising a V_{L} and V_{H} joined by a linker, e.g., a peptide linker), (scFv)2, sc(Fv)2, bispecific sc(Fv)2, bispecific (scFv)2, minibody (sc(FV)2 fused to CH3 domain), triabody is trivalent sc(Fv)3 or trispecific sc(Fv)3, (4) a multivalent antibody (an antibody comprising binding regions that bind two different epitopes or proteins, e.g., “scorpion” antibody, and (5) a fusion protein comprising a binding portion of an immunoglobulin fused to another amino acid sequence (such as a fluorescent protein). The antibody can be a monoclonal antibody or a polyclonal antibody. An antibody “specifically binds” or is “specific for” a target antigen or target group of antigens if it binds the target antigen or each member of the target group of antigens with an affinity of at least any of 1×10⁻⁶ M, 1×10⁻⁷ M, 1×10⁻⁸ M, 1×10⁻⁶ M, 1×10^{—16} M, 1×10⁻¹¹ M, 1×10⁻¹² M, and, for example, binds to the target antigen or each member of the target group of antigens with an affinity that is at least two-fold greater than its affinity for non-target antigens to which it is being compared.

Other molecules that bind to N4-acdC include, for example a naturally-occurring N4-acdC-binding protein, and proteins that have been engineered to bind to N4-acdC. One such protein is N-acetyltransferase 10 (“Nat 10”).

Binding of an antibody or other binding agent to nucleic acids comprising N4-acdC residues produce complexes that allow for purification. For example, the binding agent could be bound to a solid support, such as a particle, such as a chromatography medium or magnetically attractable beads. After binding of nucleic acids comprising N4-acdC residues to the binding agent, unbound material is removed and captured nucleic acids are eluted or released from the binding agent. Alternatively, the complexes can be captured using a secondary binding agent that binds to the primary binding agent. For example, the primary binding agent can be an IgG antibody and the secondary binding agent can be an antibody that binds IgG.

In another embodiment, where N4-acdC has been converted to N4-acetyl-3,4,5,6-tetrahydrocytidine (“N4-athC”), one can generate and use an antibody that recognizes N4-athC.

### E. Isolation Of Nucleic Acids Comprising N4-AcdC Residues by Derivatizing With The Tag

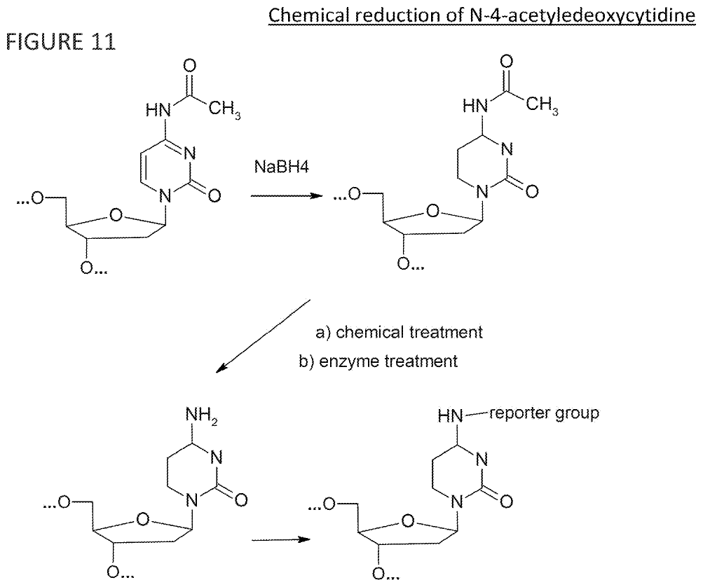

Referring to FIG. 11, another method for enriching for nucleic acids comprising N4-acdC residues involves derivatizing the residues with a tag that can be captured by a binding agent. For example, a strong chemical reducing agent, such as sodium borohydride, can convert N4-acetyldeoxyCytidine to N4-acetyl-3,4,5,6-tetrahydrocytidine. Enzymatic deacetylation of the N4-acetyl moiety yields a nucleophilic primary amine that is then amenable to a range of standard bioconjugation chemistries (e.g., labeling with N-hydroxysuccinimidylester functionalized dyes, biotin, etc.). The derivatized molecules can then be captured using a binding moiety for the tag. For example, if the tag is biotin, the capture moiety can be streptavidin.

### III. Mapping Strategies

Nucleic acids enriched for molecules comprising N4-acdC residues can then be subject to analysis.

Strategies for mapping N4-acdC residues in DNA molecules can involve methods that compare samples enriched for DNA with N4-acdC residues and samples not enriched in the same manner. Strategy also can involve converting non-N4-acdC residues into a different form, such as uracil, that can be differentiated upon sequencing. In this case, upon sequencing, N4-acdC residues will read out as C, while other forms of cytosine will read out as T. Alternatively, N4-acdC residues can be converted into a different form, such as uracil. In this case, upon sequencing, N4-acdC residues will read out as T, while other forms of cytosine will read out as C.

Because N4-acdC residues in DNA have significant overlap with G4 structures, the methods provided herein are useful for mapping G4 structures as well.

### A. DNA Immunoprecipitation Sequencing

In one embodiment mapping of N4-acdC residues in nucleic acid molecules involves comparing a first aliquot of the sample in which N4-acdC residues have been removed with a second aliquot in which they have not.

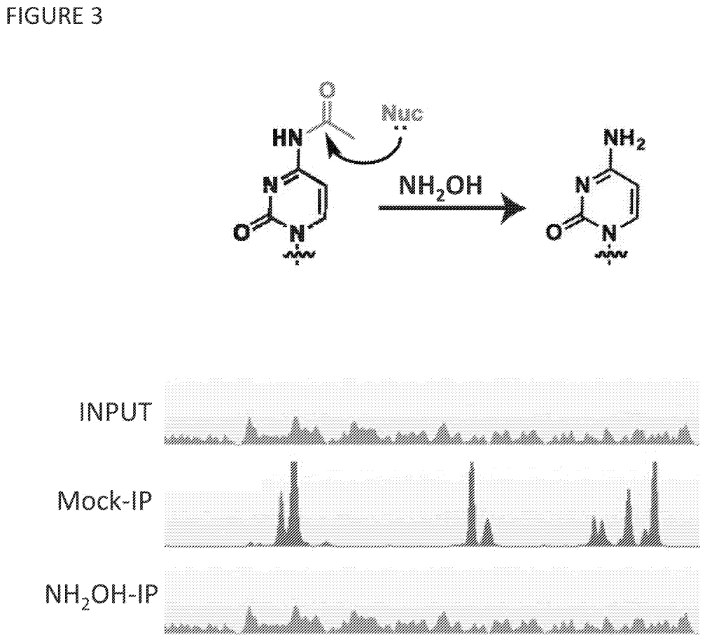

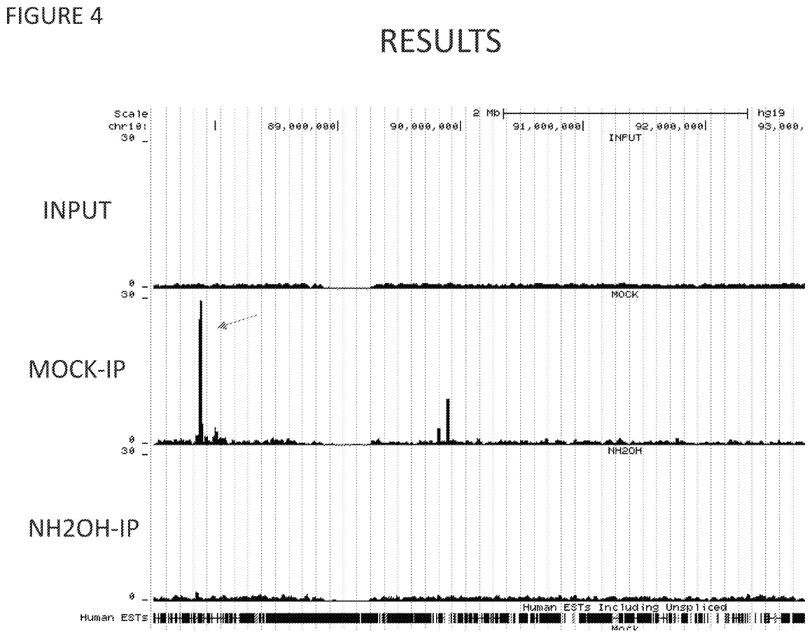

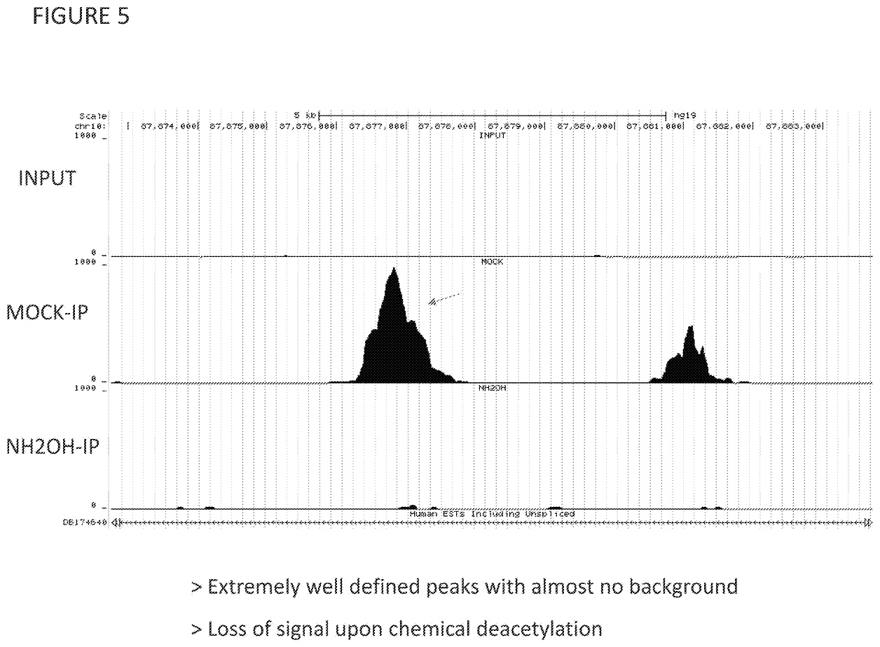

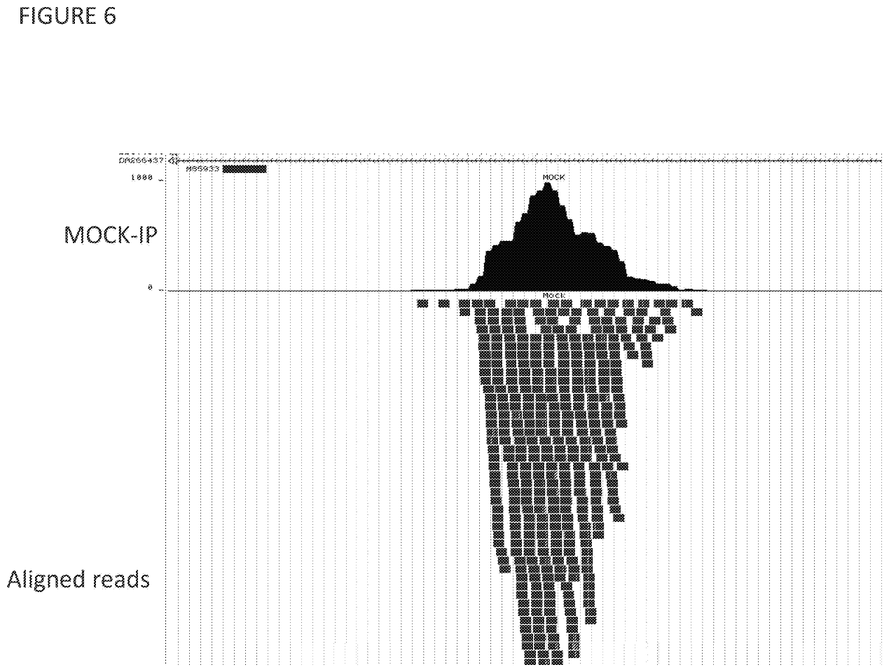

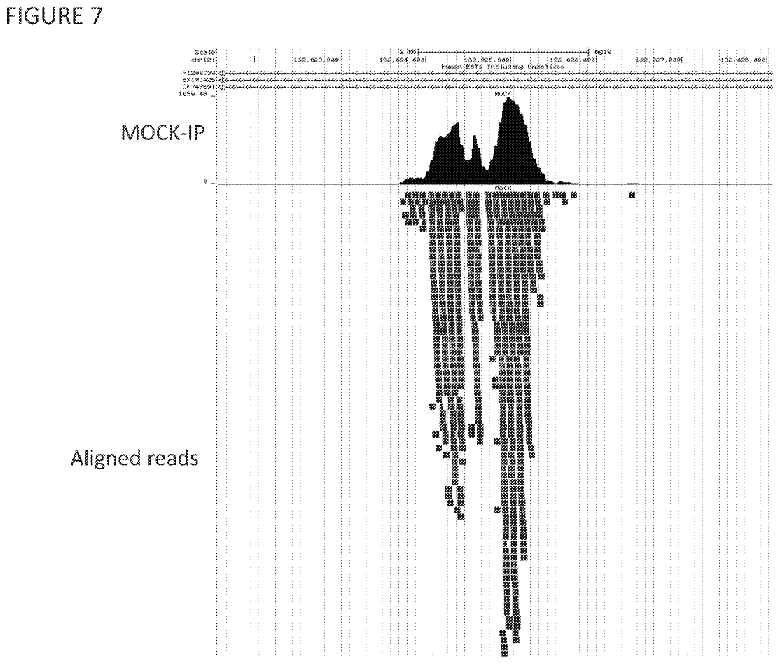

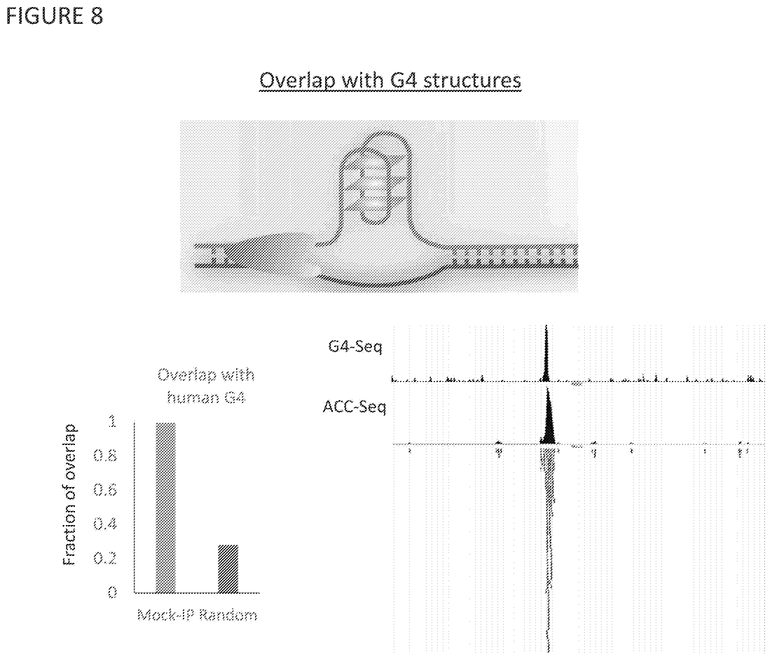

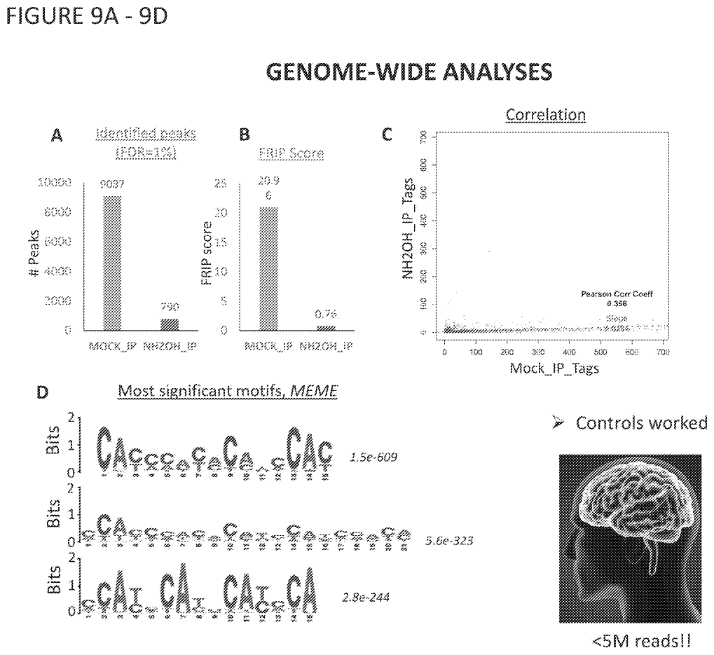

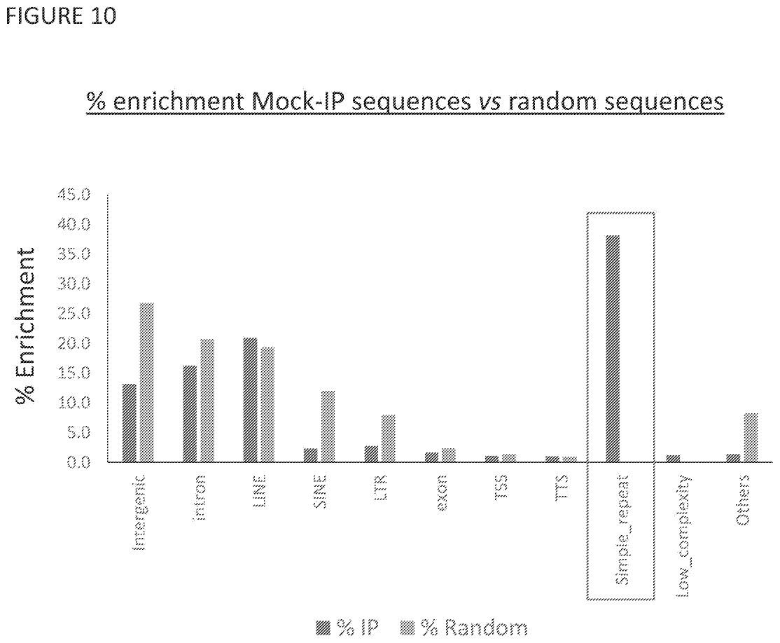

According to such methods N4-acdC residues in a first aliquot of the sample are deacetylated to cytidine residues (See, e.g., FIG. 3.). N4-acdC residues can be converted to cytidine using a nucleophile. The nucleophile can be, for example, hydroxylamine, sodium hydroxide, or NH₄OH/CH₃NH₂ (Ammonium Hydroxide/aqueous MethylAmine or AMA reagent) reagent. Sodium hydroxide may also serve as a denaturing agent.

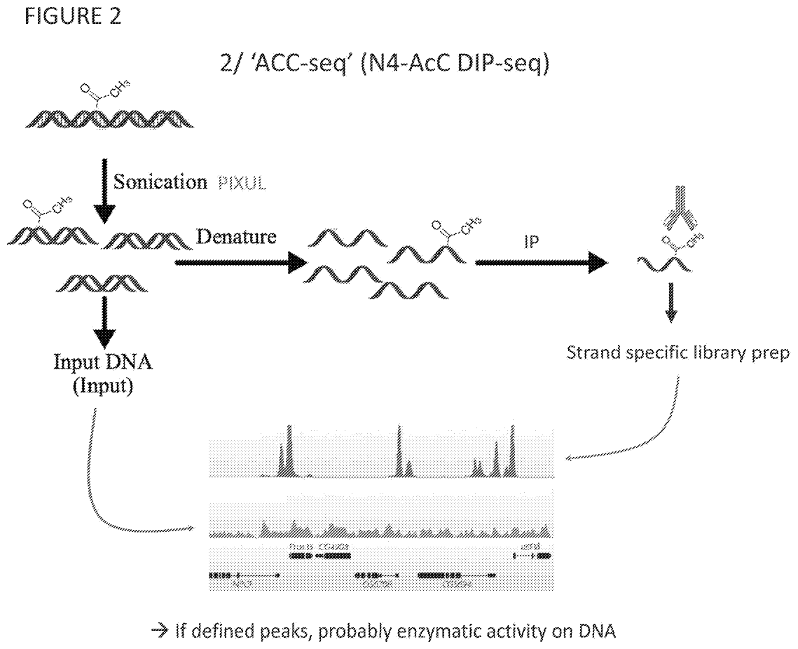

Next, each of aliquots is incubated with a binding agent that recognizes nucleic acid comprising N4-acdC residues. (See, e.g., FIG. 2.) Bound nucleic acids from each aliquot are isolated. The isolated nucleic acids from each aliquot are subjected to nucleic acid sequencing. Isolated nucleic acids from the untreated aliquot will be overrepresented by molecules containing N4-acdC residues. Accordingly, when sequence reads from both aliquots are mapped to a reference genome, at any genetic locus, the read depth from samples that comprise DNA with N4-acdC residues will be deeper than the read depth from samples in which DNA has been deacetylated. This will indicate the abundance of N4-acdC residues at these loci in the analyzed sample.

In another embodiment, cells are treated with NaBH₄ or other reducing agents to produce N4-acetyl-3,4,5,6-tetrahydrocytidine, a very stable reduced form of N4-acdC. In this case, the binding agent used would be directed against N4-acetyl-3,4,5,6-tetrahydrocytidine and not N4-acdC. The method also offers the advantage that a C-T SNP or a stop/deletion will be seen on sequencing reads at N4-acetyl-3,4,5,6-tetrahydrocytidine sites, offering a base-resolution identification of the N4-acdC in genomic DNA.

### B. Kinetic Trapping Followed by Bisulfite Sequencing

In one embodiment, cytosine residues other than N4-acdC are protected by a transamination process, for example, using bisulfite in the presence of a nucleophile. For example, using methylhydroxylamine, the position 4 amine group is converted to hydroxymethylamine.

After transamination, N4-acdC residues are deaminated, for example, using bisulfite, converting them to uracil. Transaminated cytosine and other 5′-modified cytosine such as 5mC and 5hmC are not deaminated.

Upon sequencing, former N4-acdC residues will read out as thymine.

In this method, because 5fC and 5caC also read out as a thymine upon bisulfite treatment, one needs a control sample where N4-acdC has been removed by deacetylation. This is compared to a control to the original sample for an unambiguous detection of N4-acdC.

Alternatively, one can convert 5fC to 5caC using TET enzyme or catalytic domain, then blocking 5caC with carbodiimide and a primary amine containing nucleophile, e.g., benzylamine. Next, after bisulfite treatment, 5fC and 5caC will also be read as C and only N4-acdC will be read as T.

This strategy takes advantage of the different rates of reaction between bisulfite and transaminated cytosine vs. N4-acetyldeoxyCytidine. The methods involve first reacting all unmodified cytosines with bisulfite in the presence of a nucleophile (e.g., methylhydroxylamine) to achieve a transamination reaction product that is refractory to deamination by bisulfite. These transaminated products will read out as cytosines in sequencing. Further reaction with bisulfite will result in deamination of N4-acetyldeoxyCytidine and its subsequent read-out as a T in downstream sequencing. Because 5-mC (5-methylcytosine)/5-hmC (5-hydroxymethylcytosine) are also refractory to bisulfite deamination, they will not interfere with base-resolution detection of N4-acetyldeoxyCytidine. In addition, differential chemical deacetylation of N4-acetyldeoxyCytidine will allow us to very specifically query for the presence of N4-acetyldeoxyCytidine in a genome-wide manner.

### C. Modified Methylase Assisted Bisulfite/Chemical Modification-Assisted Bisulfite Sequencing

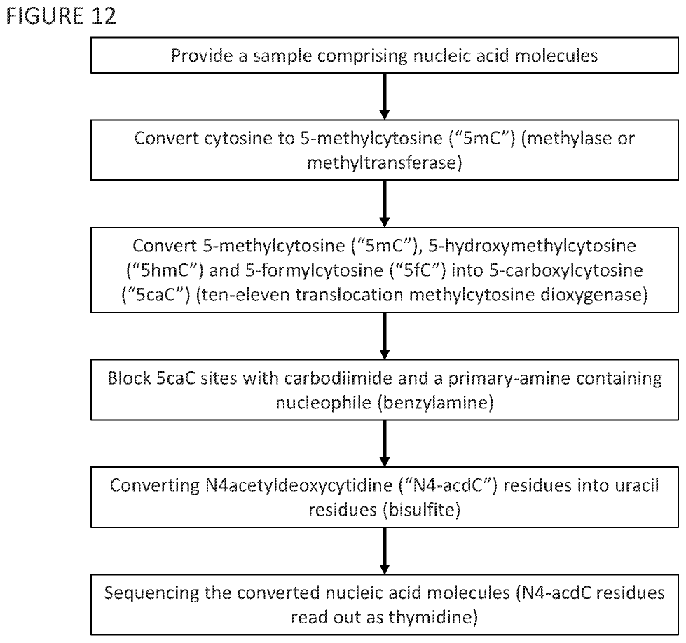

FIG. 12 shows a method of modified methylase assisted bisulfite/chemical modification-assisted bisulfite sequencing.

IV. In a first step, cytosine residues are converted into 5mC residues. This can be done, for example, by using methylase or methyltransferase. such as CpG methyltransferase (mSssl). One could also use a plant methyltransferase that can methylates CpG and non CpG sites.

Non-N4-acdC residues in the nucleic acid sample, such as 5mC, 5hmC and 5fC, are converted to 5-carboxylcytosine residues. Conversion of nucleotides to 5-carboxyl cytosine can be accomplished using TET. Ten-Eleven-Translocation methylcytosine dioxygenase (“TET”) converts 5mC, 5hmC and 5fC into 5caC. It is available from a number of different species, including human, mouse, or invertebrate (e.g., Naegleria, Drosophila (dTet, also named DMAD or CG43444)). Mammalian TET includes TET1, TET2 and TET3. The TET enzymes each harbor a core catalytic domain with a double-stranded β-helix fold that contains the crucial metal-binding residues found in the family of Fe(II)/α-KG-dependent oxygenases. These catalytic domains also can be used in conversion steps. Accordingly, “TET” refers to the whole enzyme or a functioning catalytic domain, unless otherwise specified.

5-carboxyl Cytosine residues are then blocked. Blocking can be performed with carbodiimide and a primary amine containing nucleophile, e.g., benzylamine.

Then, N4-acdC residues can be converted to uracil, for example, using bisulfite treatment.

During nucleic acid sequencing, cytosine will read out as “C” while N4-acdC residues will read out as “T”.

### A. Differential Restriction DNA Immunoprecipitation

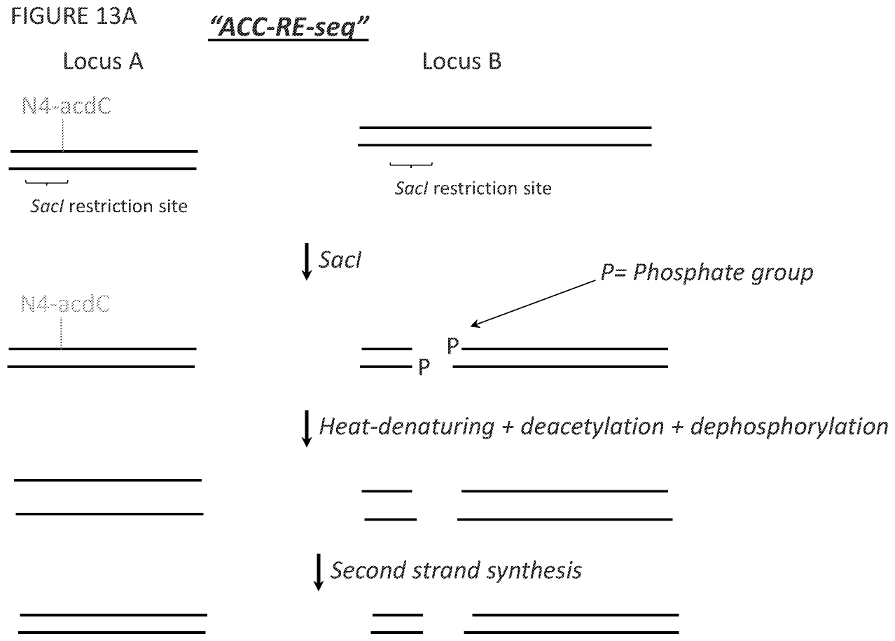

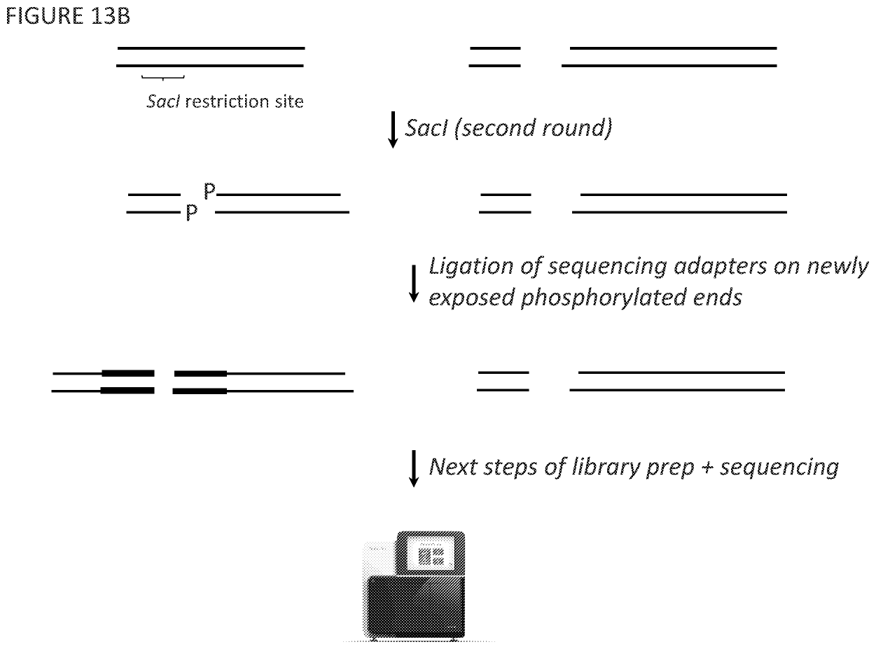

Referring to FIGS. 13A-B, in another embodiment, DNA, such as, genomic DNA, is extracted. The extracted DNA is subjected to restriction with a restriction enzyme that is does not cleave restriction sites having one or more N4-acdC residues. Such enzymes include, for example, Sacl (restriction site: GAGCTC), Kpnl, Paul and Bsh1236l. (See, e.g., Jakubovska et al. DOI: 10.1002/cbic.201900280.) Therefore, all loci (B) containing the unacetylated restriction site will be digested, exposing phosphate groups in 5′. However, loci (A) having restriction sites comprising N4-acdC residues will not be cleaved.

After DNA melting (needed for proper deacetylation), and chemical deacetylation+enzymatic dephosphorylation, a second strand of DNA is made using a DNA polymerase (e.g. Klenow, T4, etc.), reconstructing the Sacl restriction site on locus A, but not on locus B.

To perform second strand synthesis, primers are extended using an appropriate polymerase. The polymerase can be a mesophilic or thermophilic polymerase. For example, the polymerase can be Klenow exo-polymerase, Klenow polymerase, T4 DNA polymerase, Taq polymerase, pfu polymerase, DNA polymerase I and a reverse transcriptase (e.g., Moloney Murine Leukemia Virus (M-MLV), Avian Myeloblastosis Virus (AMV), and their mutated/altered versions).

Upon a second digestion, phosphates are now exposed in the previously acetylated region, enabling ligation of a sequencing adapter and further analyses of N4-acdC.

### B. Identification of Proteins Binding to N4-AcdC Residues

Also provided herein is a method for identifying a candidate protein that binds to N4-acdC in DNA, or that binds to DNA sequences containing N4-acdC. In one embodiment, the method comprises generating fragments of a DNA sample and dividing the fragments into two portions. A first portion of the DNA fragments are treated with a deacetylating agent. The second portion is not so treated. DNA from the first and second portions are then contacted with one or a plurality of proteins, which are allowed to bind to the DNA in the portions. Then, a protein or proteins that bind to DNA in the first portion are compared with amounts of proteins that bind to DNA in the second portion. A protein that binds in greater amount amounts to DNA in the second portion than the first portion is a candidate N4-acdC binding protein. Proteins can be identified by mass spectrometry.

### V. Nucleic Acid Analysis

Double-stranded nucleic acid molecules, whether amplified or not, may be subject to analysis.

### A. Library Preparation

DNA sequencing typically will involve a step of library preparation.

### 1. Isolation of Double-Stranded Nucleic Acids

Double-stranded nucleic acids may be separated from remaining single-stranded nucleic acids in a number of ways. In one embodiment, the composition can be subject to a single-strand nuclease, such as, but not limited to, nuclease S1 to digest single-stranded molecules. In another embodiment, single-stranded nucleic acids and double-stranded nucleic acids can be fractionated from one another using known methods. In one such embodiment, DNA is isolated using silica or non-silica -based methods that have high affinity for double-stranded nucleic acids and low affinity for single-stranded nucleic acids, such as silica or hydroxyapatite. These can involve binding DNA to silica particles or membranes, or DNA grade Bio-Gel HTP hydroxyapatite, and separating from other contaminants. In one embodiment, double-stranded nucleic acids can be specifically enriched by the use of double-stranded nucleic acid binding proteins such as anti-double-stranded DNA anti-idiotypic antibodies. In one embodiment, single-stranded nucleic acids can be removed (negative selection) by single-stranded nucleic acid binding proteins such as anti-single-stranded DNA anti-idiotypic antibodies. In one embodiment, primers are provided with a capture moiety such as, for example, biotin or desthiobiotin. Accordingly, double-stranded molecules created through primer extension will be biotinylated. These molecules can be isolated through capture with a partner for the capture moiety, such as streptavidin, and single-stranded DNA molecules can be digested by single-strand nuclease, such as, but not limited to, nuclease S1.

After end repair and adapter ligation, target nucleic acid sequences can be isolated using capture sequences. Capture sequences are polynucleotides comprising a nucleotide sequence capable of hybridizing to nucleic acid molecules having a target sequence. Once hybridized, the target sequences capture the hybridized sequences. Typically, probes will comprise a capture moiety, such biotin, or will be attached to a solid support, such as a magnetically attractable particle, to allow for separation of the bound material from unbound material.

### 2. End Repair and Adapter Ligation

Polynucleotides subjected to fragmentation, or cell free DNA typically comprise ends with single-stranded overhangs that require end repair before adapter ligation. End repair can be accomplished by, for example, an enzyme such as Klenow polymerase which cleaves back 5′ overhangs and fills in 3′ overhangs. The result is a blunt ended molecules. Adapters can be attached to blunt end DNA directly by blunt end ligation. Alternatively, the blunt ended molecules can be “A tailed” in the 3′ ends to produce a single nucleotide “A” overhang. Sequencing adapters having a single “T” overhang in the 5′ ends can therefore be attached.

Alternatively, as discussed above, target polynucleotides can be provided with adapters through a primer extension reaction in which a primer molecule, as described herein further comprises adapter sequences. In this instance, after elongation by a polymerase, DNA is tagged at the 3′ end with an azido-ddNTP. Then an adapter containing an alkyl 5′ can be attached by click chemistry. DNA can then be PCR-amplified and further analyzed.

In another embodiment, adapter molecules comprising hairpin loops, including methylated C residues in the double strand stem are ligated (and with no C residues in the loop), then after bisulfite and primer anchoring, a “rolling circle” -mediated library is performed using an enzyme that contains a strong displacement activity such as Phi29/ϕ29 polymerase.

### 3. Nucleic Acid Amplification

Double-stranded nucleic acids can be amplified. Amplification typically is performed on nucleic acids provided with adapters comprising primer hybridization sequences. Double-stranded nucleic acids can be amplified by any known form of amplification. This includes, without limitation, polymerase chain reaction (PCR) amplification, quantitative PCR, rolling circle amplification, multiple displacement amplification, loop-mediated isothermal amplification (LAMP), reverse transcription loop-mediated isothermal amplification (RT-LAMP), strand-displacement amplification (SDA), helicase-dependent amplification (HDA), or transcription-mediated amplification (TMA). For ease of description, reactions will be discussed in terms of PCR; necessary adjustments for other methods of amplification will be readily apparent to one of skill in the art.

### B. Nucleic Acid Sequencing

In one embodiment, double-stranded nucleic acids are analyzed by nucleic acid sequencing. Typically, nucleic acids are sequenced using high throughput sequencing. As used herein, the term “high throughput sequencing” refers to the simultaneous or near simultaneous sequencing of thousands of nucleic acid molecules. High throughput sequencing is sometimes referred to as “next generation sequencing” or “massively parallel sequencing.” Platforms for high throughput sequencing include, without limitation, massively parallel signature sequencing (MPSS), Polony sequencing, 454 pyrosequencing, Illumine (Solexa) sequencing, SOLID sequencing, Ion Torrent semiconductor sequencing, DNA nanoball sequencing, Heliscope single molecule sequencing, single molecule real time (SMRT) sequencing (PacBio), and nanopore DNA sequencing (e.g., Oxford Nanopore).

### C. Analysis of Nucleic Acid Sequences

Nucleic acid sequencing produces sequence reads. Sequence reads are typically analyzed by mapping the sequence reads to a reference genome. For example, the current human genome reference sequence is hg38, which can be accessed at, for example, the NCBl website. A genetic locus for analysis can be a single nucleotide position in the genome, or a sequence or area of the genome, such as a gene, including surrounding areas such as promoter regions, or a chromosome.

After mapping sequences to a reference genome the results can be analyzed in a number of ways. One method of analysis is referred to as “peak analysis”. In this method the number of sequence reads mapping to loci across the reference genome can be determined. Because the nucleic acids have been enriched for sequences comprising modified nucleotides, loci to which many sequence reads appear as “peaks” of reads, for example, in a graph in which the X axis represents the genome and the “Y” axis represents the number of reads mapping thereto. Peaks can represent loci of nucleotide modification.

Another method involves single base resolution analysis. In this method, sequence reads are compared against a reference genome, using a single nucleotide in the reference genome as a “locus”. Cytosine form nucleotides that were converted to non-cytosine form nucleotides will appear as mismatches against the reference genome. For example, unmodified cytosine residues in the sequence read would match with a cytosine residue in the reference genome. Modified cytosine residues in the sequence reads that have been converted to uracil will mismatch cytosine residues in the reference genome.

### D. Direct Identification with Oxford Nanopore Sequencing

Nanopore sequencing (4th generation sequencing) has gained more visibility in the last years since it is one of the few methods that can identify—directly via sequencing—DNA modifications. This strategy will be applied to probe N4-acetyldeoxyCytidine by differentially treating DNA with a deacetylating agent (e.g. NH2OH/NaOH), a reducing agent (e.g. NaBH4), or any bulky adduct that can specifically be attached to N4-acetyldeoxyCytidine, then sequencing DNA with Oxford Nanopore. Differential treatment will produce current/voltage variations that can be used to identify the modified base.

### E. DNA Microarray Analysis

In some embodiments, nucleic acids prepared by the methods described herein can be analyzed using a DNA microarray. DNA microarrays can be used for comparative genomic hybridization, chromatin immunoprecipitation analysis, and SNP detection. DNA micorarrays, also referred to as “DNA chips” are solid supports to which are attached positionally defined and addressable oligonucleotide probes. When sample nucleic acids are contacted with the array of nucleic acid probes, the sample nucleic acids hybridize to probes having complementary, or nearly complementary, sequences. The locations where sample nucleic acids have hybridized can be determined. This information can then be used to determine the identity or the sequence of the sample nucleic acids. Because they can detect nucleic acid molecules in a sequence-specific manner, DNA microarrays are useful for detecting sequences altered such that bases that read as “C” in a reference genome, are replaced by “T” after being treated by the methods described herein. DNA microarrays can be prepared in the lab, or purchased from, for example, Affymetrix (ThermoFisher).

### VI. Diagnostic Methods

The location of N4-acdC residues in DNA molecules can be used in diagnostic methods that involve detection of modified bases as biomarkers. In methods of discovering biomarkers, samples from two groups of subjects, one with a condition to be diagnosed, and the other without the condition, are provided. The condition can be any pathological condition including, without limitation, genetic conditions, cancers, age-related conditions such as progeria or accelerated aging, cellular pathologies, neuronal pathologies, etc.

Methods as described herein are used to produce genetic analysis of base modification patterns in each of the samples of each of the different groups. This genetic analysis can take the form of sequence information. The data is collected into a dataset and subject to statistical analysis to generate a model that distinguishes between the two groups. Any statistical method known in the art can be used for this purpose. Such methods, or tools, include, without limitation, correlational, Pearson correlation, Spearman correlation, chi-square, comparison of means or variance (e.g., paired T-test, independent T-test, ANOVA) regression analysis (e.g., simple regression, multiple regression, linear regression, non-linear regression, logistic regression, polynomial regression, stepwise regression, ridge regression, lasso regression, elastic net regression) or non-parametric analysis (e.g., Wilcoxon rank-sum test, Wilcoxon sign-rank test, sign test). Such tools are included in commercially available statistical packages such as MATLAB, JMP Statistical Software and SAS. Such methods produce models or classifiers which one can use to classify a particular biomarker profile into a particular state. Statistical analysis can be operator implemented or implemented by machine learning. The result of such analysis is a model that uses information about the location of modified bases, e.g., modified cytosine residues, to classify a subject from which a sample is taken as having or not having the condition.

Once a model for diagnosing a condition is established, the model can be used for diagnosis of a subject. In such methods, a sample comprising nucleic acids from the subject is provided. The nucleic acids are subject to the methods as described herein. Treated nucleic acids are analyzed to generate characteristic data, such as sequence data. The model is applied to the sequence data to classify the sample into the appropriate category.

For example, the methods of detection can comprise (1) providing a DNA sample from a subject, and (2) mapping the location of N4-acdC residues in the sample to genetic loci. Analysis can be genome-wide, or can be limited to genetic loci having known N4-acdC biomarkers.

The methods can involve any of the mapping strategies described herein. This includes immunoprecipitation methods in which a sample is divided into two aliquots and one aliquot is subject to deacetylation. Alternatively, DNA from a biological sample can be subject to treatment in which N4-acdC residues are converted to uracil (e.g. bisulfite sequencing). Upon mapping, uracil residues will map to cytosine residues in a reference genome, thereby indicating the presence of N4-acdC residues in the biological sample. Furthermore, detection can be done by any method known in the art for detecting particular nucleotide sequences, including, but not limited to DNA sequencing, PCR, qPCR, hybridization of labeled probes against the biomarker, TaqMan amplification, or detection by molecular beacon.

### VII. Compositions

The presence of N4-acdC residues in naturally occurring DNA molecules is a new discovery. As such, provided herein are compositions of matter comprising DNA molecules comprising N4-acdC residues.

In another embodiment, provided herein are DNA molecules comprising N4-athC residues, optionally purified.

In one embodiment, provided herein are compositions comprising a complex between a binding agent that specifically binds the acetyl group of N4-acdC and DNA molecules comprising N4-acdC residues. Such compositions can comprise naked DNA or DNA in the form of chromatin. The complexes can be enriched or isolated from normally present cellular macromolecules such as any of proteins, complex carbohydrates or lipids. In another embodiment DNA molecules comprising N4-acdC molecules are enriched compared to a comparable naturally occurring sample by a factor of at least two, at least 10 or at least 100.

### VIII. Kits

As used herein, the term “kit” refers to a collection of items intended for use together. Such items can be packaged in a single container. The kit can optionally include instructions for use thereof. A kit can further include a shipping container adapted to hold a container, such as a vial, that contains a composition as disclosed herein.

Kits provided herein can include a first container containing a deacetylating agent, such as a nucleophile, e.g., hydroxylamine, sodium hydroxide, or NH₄OH/CH₃NH₂ (Ammonium Hydroxide/aqueous MethylAmine or AMA reagent) reagent, and a second container containing a binding agent that specifically binds DNA comprising N4-acdC residues or N4-athC residues, as the case may be.

In another embodiment, a kit comprises a first container containing a deacetylating agent, and a second container containing a restriction enzyme that does not recognize restriction sites having at least one acetylated nucleotide.

In another embodiment, a kit comprises a first container containing a deacetylating agent, a second container containing a reducing agent, a third container containing a deacetylase, a fourth container containing a molecular tag, and, optionally, a fifth container containing a binding agent that binds the tag.

In another embodiment, a kit comprises a first container containing a deacetylating agent, a second container containing bisulfite reagent, and a third container containing a TET enzyme or catalytic domain.

In another embodiment, a kit comprises a first container containing a deacetylating agent, and a second container containing a polymerase, such as BSM polymerase. The kit also can include a container comprising other elements for library preparation, such as oligonucleotide adapters. In certain instances, N4-acdC will itself induce a SNP, such as C4T conversion.

### EXEMPLARY EMBODIMENTS

Exemplary embodiments of the invention include, but are not limited to:

1. A method for obtaining a population of DNA fragments containing N4-acetydeoxycytine (N4-acdC), the method comprising:
(a) generating DNA fragments from DNA in a sample;
(b) contacting the DNA fragments with a N4-acdC binding agent; and
(c) enriching for complexes between the binding agent and the DNA fragments.

2. A method comprising:
a) providing a sample comprising nucleic acid molecules;
b) converting N4-acetyldeoxycytidine (“N4-acdC”) residues in the nucleic acid molecules into N4-acetyl-3,4,5,6-tetrahydrocytidine residues;
c) deacetylating the N4-acetyl-3,4,5,6-tetrahydrocytidine residues to produce primary nucleophilic amines;
d) conjugating the primary nucleophilic amines with a tag;
e) capturing nucleic acids attached to the tag from the sample using a capture molecule;
f) sequencing captured nucleic acid molecules to produce sequence data comprising sequencing reads.

3. The method of embodiment 2, wherein converting N4-acetyldeoxycytidine (“N4-acdC”) residues in the nucleic acid molecules into N4-acetyl-3,4,5,6-tetrahydrocytidine residues comprises the use of a reducing agent (e.g., NaBH4, LiBH4, KBH4, NBu4BH4, NaCNBH3, BH3-pyr).

4. The method of embodiment 2, wherein deacetylating is performed chemically or enzymatically.

5. The method of embodiment 2, wherein the tag comprises biotin or desthiobiotin.

6. The method of embodiment 2, wherein capture molecule comprises avidin, streptavidin, or NeutrAvidin.

7. A method comprising:
(a) preparing a first aliquot and a second aliquot from a sample comprising nucleic acid molecules;
(b) in the first aliquot, converting N4acetyldeoxycytidine (“N4-acdC”) residues in nucleic acid molecules into cytidine residues to produce a first deacetylated aliquot;
(c) contacting a nucleic acids in the first deacetylated aliquot and in the second aliquot with a N4-acdC binding agent; and
(d) enriching for complexes between the binding agent and the nucleic acid molecules comprising N4-acdC residues in the first deacetylated aliquot and in the second aliquot to produce enriched first and second aliquots; and
(e) isolating nucleic acids in the enriched aliquots.

8. The method of embodiment 2, further comprising:
(f) sequencing isolated nucleic acid molecules from both enriched aliquots to produce sequence data comprising sequencing reads;
(g) measuring sequence reads from each enriched aliquot that map to one or a plurality of genetic loci;
(h) identifying one or more genetic loci in which the measure of mapped sequence reads from the second aliquot is greater than the measure of mapped sequence reads from the deacetylated aliquot, wherein genetic loci at which the measure of mapped sequence reads is greater for the second aliquot than for the deacetylated aliquot represent loci of nucleic acid in the sample comprising N4-acdC.

9. The method of embodiment 2, wherein converting comprises treating the nucleic acids with a nucleophile.

10. The method of embodiment 9, wherein the nucleophile comprises hydroxylamine, sodium hydroxide, or NH4OH/CH3NH2 (Ammonium Hydroxide/aqueous MethylAmine or AMA reagent) reagent.

11. The method of embodiment 10, wherein NaOH also serves as a denaturing agent.

12. The method of embodiment 1 and 7, wherein the nucleic acid molecules are immunoprecipitated with an anti-N4-acdC or anti-N4-acetylcitidine (“4N-AcC”) antibody.

13. A method comprising:
a) providing a sample comprising nucleic acid molecules;
b) transaminating unmodified cytidine in the nucleic acid molecules by treating the molecules with bisulfite in the presence of a nucleophile;
c) deaminating N4-acdC residues in the nucleic acid molecules to produce uracil residues, (e.g., with bisulfite); and
d) sequencing the deaminated nucleic acid molecules, wherein N4-acdC residues will read out as thymidine;
e) optionally providing a control sample wherein, before transaminating unmodified cytidine, N4-acdC residues are deacetylated, wherein, later, N4-acdC will read out as cytosine.

14. A method comprising:
a) providing a sample comprising nucleic acid molecules;
b) transaminating unmodified cytidine in the nucleic acid molecules by treating the molecules with bisulfite in the presence of a nucleophile;
c) converting 5-methylcytosine (5mC), 5-hydroxymethylcytosine (5hmC), and 5-formylcytosine (“5fC”) into 5-carboxylcytosine (“5caC”) (using, e.g. Ten-eleven translocation methylcytosine dioxygenase);
d) blocking 5caC residues with carbodiimide and a primary amine-containing nucleophile;
e) deaminating N4-acdC residues in the nucleic acid molecules to produce uracil residues, (e.g., with bisulfite); and
f) sequencing the deaminated nucleic acid molecules, wherein N4-acdC residues will read out as thymidine.

15. A method comprising:
a) providing a sample comprising nucleic acid molecules;
b) treating the nucleic acid to convert cytosine to 5-methylcytosine (“5mC”), e.g., with a methylase or methyltransferase such as mSssl;
c) converting 5-methylcytosine (“5mC”), 5-hydroxymethylcytosine (“5hmC”) and 5-formylcytosine (“5fC”) into 5-carboxylcytosine (“5caC”) residues, e.g., by treatment with a ten-eleven translocation methylcytosine dioxygenase (“TET”);
d) blocking 5caC sites with carbodiimide and a primary-amine containing nucleophile, e.g., benzylamine; and
e) converting N4acetyldeoxycytidine (“N4-acdC”) residues into uracil residues using bisulfite; and
f) sequencing the converted nucleic acid molecules, wherein N4-acdC residues will read out as thymidine.

16. The method of embodiment 15, wherein cytosine is converted to 5mC chemically or with a CpG methyltransferase (e.g. from bacteria or plants).

17. The method of embodiment 15, wherein the TET is selected from TET1, TET2, TET3, mouse TET, drosophila TET (CG43444), and NgTET (Naegleria Tet-like dioxygenase).

18. A method comprising:
(a) digesting DNA comprising N4-acdC residues with a restriction enzyme, wherein the restriction enzyme does not digest restriction sites comprising N4-acdC (e.g., Sac l)
(b) denaturing, deacetylating and dephosphorylating the digested DNA;
(c) performing second strand synthesis on the denatured, deacetylated and dephosphorylated DNA to produce double-stranded DNA molecules;
(d) digesting double-stranded DNA from operation (c) with the restriction enzyme leaving cleaved, phosphorylated molecules;
(e) attaching adapter molecules to the cleaved, phosphorylated molecules to produce adapter-tag molecules; and
(f) optionally, amplifying by PCR and sequencing the adapter-tag molecules.

19. A method comprising:
a) providing a sample comprising nucleic acid molecules;
b) treating N4-acetyldeoxycytidine (“N4-acdC”) residues in the nucleic acids with a deacetylating agent, a reducing agent, or by attaching an adduct; and
c) sequencing the treated nucleic acid molecules by nanopore sequencing, wherein treated N4-acdC residues produce a characteristic variation in current or voltage during sequencing.

20. A method for identifying a candidate protein that binds to N4-acdC in DNA, or that binds to DNA sequences containing N4-acdC, the method comprising:
(a) generating fragments of a DNA sample;
(b) contacting a first portion of the DNA fragments, but not the second portion, with a deacetylating agent;
(c) contacting DNA from the first and second portions with test proteins; and
(d) comparing amounts of proteins that bind to DNA in the first portion with amounts of proteins that bind to DNA in the second portion; wherein a protein that binds in greater amount amounts to DNA in the second portion than the first portion is a candidate N4-acdC binding protein.

21. The method of embodiment 20, wherein the proteins are identified by mass spectrometry.

22. A method for identifying a biomarker for a condition, the method comprising:
(a) obtaining a first set of DNA samples from each of a plurality of subjects having the condition;
(b) obtaining second set of DNA samples from each of a plurality of subjects not having the condition;
(c) generating fragments of the first and second DNA sample sets;
(d) separately contacting the fragments from each of the first and second DNA sample sets with a N4-acdC binding agent;
(e) enriching complexes between the binding agent and the DNA fragments in the first and second DNA sample sets; and
(f) determining nucleotide sequences of the DNA fragments in the complexes obtained from the first and second DNA sample sets;
wherein a DNA sequence that is more abundant in complexes formed with the first DNA sample set, compared to the second DNA sample set, is a biomarker for the condition.

23. The method of embodiment 20, wherein the condition is selected from cancer, an infectious disease, or a hereditary disease.

24. The method of embodiment 20, wherein the DNA sample is obtained from a tumor, a bodily fluid, a tissue or an organ.

25. The method of embodiment 24, wherein the bodily fluid is blood, plasma, serum, saliva, sputum, mucus, lymphatic fluid, urine, semen, cerebrospinal fluid or amniotic fluid.

26. The method of embodiment 20, wherein the DNA sample contains cell-free DNA (cfDNA).

27. The method of embodiment 20, wherein the DNA fragments are obtained by sonication, shearing or enzymatic fragmentation.

28. The method of embodiment 20, wherein the DNA sample is chromatin.

29. The method of embodiment 20, wherein the DNA sample is naked DNA.

30. The method of embodiment 29, wherein the DNA sample is double-stranded.

31. The method of embodiment 29, wherein the DNA sample is single-stranded.

32. The method of embodiment 31, further comprising, either before or after step (a), denaturing the DNA.

33. The method of embodiment 20, wherein the N4-acdC binding agent is
(a) an antibody,
(b) a naturally-occurring N4-acdC-binding protein, or
(c) a protein that has been engineered to bind to N4-acdC.

34. The method of embodiment 20, wherein the enrichment is achieved by immunoprecipitation, affinity chromatography, gel filtration or gel retardation.

35. The method of embodiment 20, wherein the N4-acdC binding agent comprises biotin or desthiobiotin.

36. The method of embodiment 35, wherein the enrichment is achieved using avidin, streptavidin or NeutrAvidin.

37. A method comprising:
(a) providing a biological sample comprising DNA from a subject; and
(b) mapping N4-acdC residues to one or more genetic loci in the DNA.

38. The method of embodiment 37, wherein mapping comprises converting N4-acdC residues in the DNA into uracil residues and identifying one or more genetic loci represented by “C” in a reference genome but represented by “T” in the DNA from the subject.

39. The method of embodiment 37, wherein mapping comprises converting N4-acdC residues in the DNA into uracil residues and identifying one or more genetic loci represented by “C” in a reference genome but represented by “T” in the DNA from the subject.

40. The method of embodiment 37, wherein mapping comprises converting N4-acdC residues in the DNA into uracil residues and identifying one or more genetic loci represented by “C” in a reference genome but represented by “T” in the DNA from the subject.

41. The method of embodiment 37, wherein mapping comprises converting non-N4-acdC residues in the DNA into uracil residues and identifying one or more genetic loci represented by “C” in a reference genome and represented by “C” in the DNA from the subject.

42. The method of embodiment 37, wherein mapping comprises:
dividing the sample into first and second aliquots;
deacetylating the DNA in the first aliquot;
optionally, immunoprecipitating DNA in the two aliquots using a binding agent that specifically binds N4-acdC residue;
sequencing the DNA in the deacetylated first aliquot and in the second aliquot; and mapping sequence reads to a reference genome, wherein genetic loci in which sequence reads are deeper for the second aliquot and then for the first acetylated aliquot represent the presence of N4-acdC.

43. The method of embodiment 37, wherein detecting comprises DNA sequencing, PCR, qPCR, hybridization of labeled probes against the biomarker, TaqMan amplification, or detection by molecular beacon.

44. The method of embodiment 37, wherein the one or more loci are biomarkers for a condition determined, for example by the method of embodiment 22.

45. The method of any of embodiments 1-46, wherein the DNA sample is obtained from a eukaryotic cell, a prokaryotic cell, an archaeal cell, a cell line, a tissue, an organ or a bodily fluid.

46. The method of embodiment 37, wherein the bodily fluid is blood, plasma, serum, saliva, sputum, mucus, lymphatic fluid, urine, semen, cerebrospinal fluid or amniotic fluid.

47. The method of any of embodiments 1-46, wherein the DNA sample contains cell-free DNA.

48. The method of any of embodiments 1-46, wherein the DNA fragments are obtained by sonication, shearing or enzymatic fragmentation.

49. The method of any of embodiments 1-46, wherein the DNA sample comprises chromatin.

50. The method of any of embodiments 1-46, wherein the DNA sample comprises naked DNA.

51. The method of embodiment 50, wherein the DNA sample comprises double-stranded DNA.

52. The method of embodiment 50, wherein the DNA sample comprises single-stranded DNA.

53. The method of embodiment 52, further comprising, either before or after step (a), denaturing the DNA.

54. The method of any of embodiments 1-46, wherein the N4-acdC binding agent is:
(a) an antibody,
(b) a naturally-occurring N4-acdC-binding protein, or
(c) a protein that has been engineered to bind to N4-acdC.

55. The method of embodiment 54, wherein enriching comprises immunoprecipitation, affinity chromatography, gel filtration or gel retardation.

56. The method of any of embodiments 1-46, wherein the N4-acdC binding agent comprises biotin or desthiobiotin.

57. The method of embodiment 54, wherein the enrichment is achieved using avidin, streptavidin or NeutrAvidin.

58. The method of any of embodiments 1-46, wherein one or more of the DNA fragments containing N4-acetydeoxycytine (N4-acdC) also contains a G-quadruplex.

59. The method of any of embodiment 1-58, comprising converting N4-acdC residues into N4-athC residues by reduction, and, as necessary, using an antibody or a protein against N4-athC rather than N4-acdC.

60. A composition comprising a DNA molecule bound to a N4-acdC binding agent.

61. The composition of embodiment 60, wherein the DNA molecules are purified from RNA and/or cytoplasmic proteins.

62. The composition of embodiment 60, wherein the N4-acdC binding agent is an antibody that specifically binds N4-acdC residues.

63. The composition of embodiment 62, wherein the antibody is labeled.

64. The composition of embodiment 63, wherein the label comprises a capture moiety (e.g., biotin) or a detectable moiety (e.g., a fluorescent molecule).

65. A composition comprising DNA molecules enriched for N4-acdC residues or N4-athC residues, wherein enrichment is at least 2×, at least 10× or at least 100× compared with a control nucleic acid from the same species as the DNA molecules.

66. A kit comprising:
(a) a first container containing a deacetylating agent; and
(b) a second container containing a binding agent that specifically binds DNA comprising N4-acdC residues.

67. A kit comprising:
(a) a first container containing a deacetylating agent; and
(b) a second container containing sodium bisulfite.

68. The kit of embodiment 67, further comprising:
(c) a third container containing a nucleophile

69. The kit of embodiment 67, further comprising: p1 (d) a third container containing a TET enzyme.

70. A kit comprising:
(a) a first container containing a methylase;
(b) a second container containing a TET enzyme;
(c) a third container containing sodium bisulfite.

71. The kit of embodiment 70, further comprising:

(d) a fourth container containing a deacetylating agent.

72. A kit comprising a first container containing a deacetylating agent, and a second container containing a restriction enzyme that does not recognize restriction sites having at least one acetylated nucleotide and, optionally, a third container containing a phosphatase enzyme.

73. A kit comprising a first container containing a deacetylating agent, a second container containing a reducing agent, a third container containing a deacetylase, a fourth container containing a molecular tag, and, optionally, a fifth container containing a binding agent that binds the tag.

74. A kit comprising a first container containing a deacetylating agent, and a second container containing a polymerase.

75. A kit comprising a first container containing a reducing agent, a second container containing an antibody or a protein that binds N4-athC.

### EXAMPLES

### I. ACC-Seq

In ACC-Seq one sample of DNA (derived from the tissues, cell lines, treated cells, etc.) is divided in two: (1) one treated with a strong nucleophile, such as hydroxylamine or sodium hydroxide, or AMA. (2) The other sample serves as a control or reference (“Mock”-treated sample). In the nucleophile-treated group, the acetyl moiety is removed to yield cytosine, thus removing the epitope recognized by the N4-acetylcytidine/N4-acetyldeoxyCytidine specific antibody. Thereafter, both the treated group and the untreated group undergo immunoprecipitation with a N4-acetylcytidine/N4-acetyldeoxyCytidine specific antibody. Immunoprecipitated DNA from both treatment groups is then purified and NGS sequencing libraries are prepared for analysis. The resulting sequencing data reveals peaks indicative of where N4-acetyldeoxyCytidine is localized in the genome; if these peaks are found in the untreated group but are not present, or reduced, in the hydroxylamine—or NaOH— treated group, they are considered real N4-acetyldeoxyCytidine—containing loci. This method is different from and improves on the known RNA method in that the relative insensitivity of DNA to base-mediated hydrolysis compared to RNA enables use of strong nucleophilic bases, like sodium hydroxide, to achieve comprehensive deacetylation of N4-acdC sites throughout the genome; use of NaOH with RNA is not possible due to the chemical lability of RNA.

Detailed steps:
Harvest 70% confluent HeLa cells, and extraction of gDNA using Qiagen DNeasy kit+add RNaseA during extraction.
Measure nanodrop concentration and 260/280 ratio.
PIXUL Sonication of 200 μg of gDNA: 20 μg/well containing 100 uL Tris-HCl pH8.0 10 mM.

Continued steps: Parameters:
Pulse: 50N
PRF:1 Khz
Process time: 30′
Burst rate:20 Hz

Continued steps:
Tapestation HS D-1000: profiles between 200 and 700 bp
Treat 500 uL (100 μg) with NH2OH: heat at 98C for 5′, then 4 C for 5′ and keep on ice.
Weight 173 mg NH2OH and add 10 mL 1M Tris HCl pH 8.0
Add 125 μL of this solution to 500 μL of gDNA that was heat-denatured.
Heat at 65C for 1h00 (deacetylation occurs), then cool at 4C.
Acetate-EtOH precipitation, with 2 washings with 1 mL EtOH 75%.
Resuspend pellet in 100 μL Tris HCl pH 7.5 10 mM.
Denature as well 100 μg non-treated gDNA (Mock' sample).
IP with 60 μg of Mock or NH2OH -treated gDNAs:
60 μg in 409 μL of Tris HCl pH 8.0 10 mM
Prepare 591 μL (for each condition) containing 1×PBS (final concentration in 1 mL), 0.05% Triton X-100 (final concentration in 1 mL), and 0.1% BSA (final concentration in 1 mL). Add this solution to the DNA.
Add 7.5 μg of anti N4-AcdC ab from Abcam (#Ab252215)=13.5 uL.
Rotate @4C for 3h 00.
After 3h00, add 75 μL of Protein G Dynabeads that were washed twice in 1 mL cold 1×PBS and resuspended in 50 μL 1×PBS.
Incubate 1h00-2h00 @4C on rotator, then remove and keep the supernatant.

Washings 5× with 1 mL of cold washing buffer (PBS 1×, 0.05% Triton X-100, 0.1% BSA).

Elute in 150 μL of elution buffer (EB=50 mM Tris HCl pH 8.0, 75 mM NaCl, 6.25 mM EDTA, 1% SDS, 7 μL of Proteinase K from Active Motif) at 800RPM/37C. Note: elute in DNA LoBind tubes.
Harvest the supernatant and purify with 1.5× Ampure Beads.
Library prep using Swift 1s protocol.
75 cycles, single end, at least 10 M reads/sample, Illumine sequencing.

Sequencing data is presented for the complete ACC-Seq method in the accompanying drawings. This data reveals very strong enrichment of N4-acetyldeoxycytidine from human DNA.

### II. Chemical Reduction Followed By Enzymatic Deacetylation

This strategy utilizes a strong chemical reducing agent, such as sodium borohydride (NaBH4), to convert N4-acetyldeoxyCytidine to N4-acetyl-3,4,5,6-tetrahydrocytidine. Enzymatic deactylation of the N4-acetyl moiety yields a nucleophilic primary amine that is then amenable to a range of standard bioconjugation chemistries (e.g., labeling with N-hydroxysuccinimidylester functionalized dyes, biotin, etc.).

Steps:

1.) React DNA with 100 mM NaBH4 for 1 hr at 37° C.

2.) Purify DNA with Active Motif's ChIP IP DNA Purification Kit or sodium acetate/ethanol precipitation to remove all reactants.

3.) Incubate NaBH4-reduced DNA with recombinant HDAC or sirtuin deacetylases (e.g., SIRT1)

4.) Purify DNA with Active Motif's ChIP IP DNA Purification Kit or sodium acetate/ethanol precipitation to remove all reactants.

5.) React DNA with NHS-LC-Biotin (Pierce Chemical) in 10 mM HEPES buffer, pH 7.5 to label cytidine N4 primary amines with biotin for subsequent enrichment with streptavidin conjugated resin.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

What is claimed is:

1. A method for obtaining a population of DNA fragments containing N4-acetydeoxycytine (N4-acdC), the method comprising:
(a) generating DNA fragments from DNA in a sample;
(b) contacting the DNA fragments with a N4-acdC binding agent; and
(c) enriching for complexes between the binding agent and the DNA fragments.

2. A method comprising:
a) providing a sample comprising nucleic acid molecules;
b) converting N4-acetyldeoxycytidine (“N4-acdC”) residues in the nucleic acid molecules into N4-acetyl-3,4,5,6-tetrahydrocytidine residues;
c) deacetylating the N4-acetyl-3,4,5,6-tetrahydrocytidine residues to produce primary nucleophilic amines;
d) conjugating the primary nucleophilic amines with a tag;
e) capturing nucleic acids attached to the tag from the sample using a capture molecule;
f) sequencing captured nucleic acid molecules to produce sequence data comprising sequencing reads.

3. The method of claim 2, wherein converting N4-acetyldeoxycytidine (“N4-acdC”) residues in the nucleic acid molecules into N4-acetyl-3,4,5,6-tetrahydrocytidine residues comprises the use of a reducing agent (e.g., NaBH4, LiBH4, KBH4, NBu4BH4, NaCNBH3, BH3-pyr).

4. The method of claim 2, wherein deacetylating is performed chemically or enzymatically.

5. The method of claim 2, wherein the tag comprises biotin or desthiobiotin.

6. The method of claim 2, wherein capture molecule comprises avidin, streptavidin, or NeutrAvidin.

7. A method comprising:
(a) preparing a first aliquot and a second aliquot from a sample comprising nucleic acid molecules;
(b) in the first aliquot, converting N4acetyldeoxycytidine (“N4-acdC”) residues in nucleic acid molecules into cytidine residues to produce a first deacetylated aliquot;
(c) contacting a nucleic acids in the first deacetylated aliquot and in the second aliquot with a N4-acdC binding agent; and
(d) enriching for complexes between the binding agent and the nucleic acid molecules comprising N4-acdC residues in the first deacetylated aliquot and in the second aliquot to produce enriched first and second aliquots; and
(e) isolating nucleic acids in the enriched aliquots.

8. The method of claim 2, further comprising:
(f) sequencing isolated nucleic acid molecules from both enriched aliquots to produce sequence data comprising sequencing reads;
(g) measuring sequence reads from each enriched aliquot that map to one or a plurality of genetic loci;
(h) identifying one or more genetic loci in which the measure of mapped sequence reads from the second aliquot is greater than the measure of mapped sequence reads from the deacetylated aliquot, wherein genetic loci at which the measure of mapped sequence reads is greater for the second aliquot than for the deacetylated aliquot represent loci of nucleic acid in the sample comprising N4-acdC.

9. The method of claim 2, wherein converting comprises treating the nucleic acids with a nucleophile.

10. The method of claim 9, wherein the nucleophile comprises hydroxylamine, sodium hydroxide, or NH₄OH/CH₃NH₂ (Ammonium Hydroxide/aqueous MethylAmine or AMA reagent) reagent.

11. The method of claim 10, wherein NaOH also serves as a denaturing agent.

12. The method of claims 1 and 7, wherein the nucleic acid molecules are immunoprecipitated with an anti-N4-acdC or anti-N4-acetylcitidine (“4N-AcC”) antibody.

13. A method comprising:
a) providing a sample comprising nucleic acid molecules;
b) transaminating unmodified cytidine in the nucleic acid molecules by treating the molecules with bisulfite in the presence of a nucleophile;
c) deaminating N4-acdC residues in the nucleic acid molecules to produce uracil residues, (e.g., with bisulfite); and
d) sequencing the deaminated nucleic acid molecules, wherein N4-acdC residues will read out as thymidine;
e) optionally providing a control sample wherein, before transaminating unmodified cytidine, N4-acdC residues are deacetylated, wherein, later, N4-acdC will read out as cytosine.

14. A method comprising:
a) providing a sample comprising nucleic acid molecules;
b) transaminating unmodified cytidine in the nucleic acid molecules by treating the molecules with bisulfite in the presence of a nucleophile;
c) converting 5-methylcytosine (5mC), 5-hydroxymethylcytosine (5hmC), and 5-formylcytosine (“5fC”) into 5-carboxylcytosine (“5caC”) (using, e.g. Ten-eleven translocation methylcytosine dioxygenase);
d) blocking 5caC residues with carbodiimide and a primary amine-containing nucleophile;
e) deaminating N4-acdC residues in the nucleic acid molecules to produce uracil residues, (e.g., with bisulfite); and
f) sequencing the deaminated nucleic acid molecules, wherein N4-acdC residues will read out as thymidine.

15. A method comprising:
a) providing a sample comprising nucleic acid molecules;
b) treating the nucleic acid to convert cytosine to 5-methylcytosine (“5mC”), e.g., with a methylase or methyltransferase such as mSssl;
c) converting 5-methylcytosine (“5mC”), 5-hydroxymethylcytosine (“5hmC”) and 5-formylcytosine (“5fC”) into 5-carboxylcytosine (“5caC”) residues, e.g., by treatment with a ten-eleven translocation methylcytosine dioxygenase (“TET”);
d) blocking 5caC sites with carbodiimide and a primary-amine containing nucleophile, e.g., benzylamine; and
e) converting N4acetyldeoxycytidine (“N4-acdC”) residues into uracil residues using bisulfite; and
f) sequencing the converted nucleic acid molecules, wherein N4-acdC residues will read out as thymidine.

16. The method of claim 15, wherein cytosine is converted to 5mC chemically or with a CpG methyltransferase (e.g. from bacteria or plants).

17. The method of claim 15, wherein the TET is selected from TET1, TET2, TET3, mouse TET, drosophila TET (CG43444), and NgTET (Naegleria Tet-like dioxygenase).

18. A method comprising:
(a) digesting DNA comprising N4-acdC residues with a restriction enzyme, wherein the restriction enzyme does not digest restriction sites comprising N4-acdC (e.g., Sac l);
(b) denaturing, deacetylating and dephosphorylating the digested DNA;
(c) performing second strand synthesis on the denatured, deacetylated and dephosphorylated DNA to produce double-stranded DNA molecules;
(d) digesting double-stranded DNA from operation (c) with the restriction enzyme leaving cleaved, phosphorylated molecules;
(e) attaching adapter molecules to the cleaved, phosphorylated molecules to produce adapter-tag molecules; and
(f) optionally, amplifying by PCR and sequencing the adapter-tag molecules.

19. A method comprising:
a) providing a sample comprising nucleic acid molecules;
b) treating N4-acetyldeoxycytidine (“N4-acdC”) residues in the nucleic acids with a deacetylating agent, a reducing agent, or by attaching an adduct; and
c) sequencing the treated nucleic acid molecules by nanopore sequencing, wherein treated N4-acdC residues produce a characteristic variation in current or voltage during sequencing.

20. A method for identifying a candidate protein that binds to N4-acdC in DNA, or that binds to DNA sequences containing N4-acdC, the method comprising:
(a) generating fragments of a DNA sample;
(b) contacting a first portion of the DNA fragments, but not the second portion, with a deacetylating agent;
(c) contacting DNA from the first and second portions with test proteins; and
(d) comparing amounts of proteins that bind to DNA in the first portion with amounts of proteins that bind to DNA in the second portion;
wherein a protein that binds in greater amount amounts to DNA in the second portion than the first portion is a candidate N4-acdC binding protein.

21. The method of claim 20, wherein the proteins are identified by mass spectrometry.

22. A method for identifying a biomarker for a condition, the method comprising:
(a) obtaining a first set of DNA samples from each of a plurality of subjects having the condition;
(b) obtaining second set of DNA samples from each of a plurality of subjects not having the condition;
(c) generating fragments of the first and second DNA sample sets;
(d) separately contacting the fragments from each of the first and second DNA sample sets with a N4-acdC binding agent;
(e) enriching complexes between the binding agent and the DNA fragments in the first and second DNA sample sets; and
(f) determining nucleotide sequences of the DNA fragments in the complexes obtained from the first and second DNA sample sets;
wherein a DNA sequence that is more abundant in complexes formed with the first DNA sample set, compared to the second DNA sample set, is a biomarker for the condition.

23. The method of claim 20, wherein the condition is selected from cancer, an infectious disease, or a hereditary disease.

24. The method of claim 20, wherein the DNA sample is obtained from a tumor, a bodily fluid, a tissue or an organ.

25. The method of claim 24, wherein the bodily fluid is blood, plasma, serum, saliva, sputum, mucus, lymphatic fluid, urine, semen, cerebrospinal fluid or amniotic fluid.

26. The method of claim 20, wherein the DNA sample contains cell-free DNA (cfDNA).

27. The method of claim 20, wherein the DNA fragments are obtained by sonication, shearing or enzymatic fragmentation.

28. The method of claim 20, wherein the DNA sample is chromatin.

29. The method of claim 20, wherein the DNA sample is naked DNA.

30. The method of claim 29, wherein the DNA sample is double-stranded.

31. The method of claim 29, wherein the DNA sample is single-stranded.

32. The method of claim 31, further comprising, either before or after step (a), denaturing the DNA.

33. The method of claim 20, wherein the N4-acdC binding agent is
(a) an antibody,
(b) a naturally-occurring N4-acdC-binding protein, or
(c) a protein that has been engineered to bind to N4-acdC.

34. The method of claim 20, wherein the enrichment is achieved by immunoprecipitation, affinity chromatography, gel filtration or gel retardation.

35. The method of claim 20, wherein the N4-acdC binding agent comprises biotin or desthiobiotin.

36. The method of claim 35, wherein the enrichment is achieved using avidin, streptavidin or NeutrAvidin.

37. A method comprising:
(a) providing a biological sample comprising DNA from a subject; and
(b) mapping N4-acdC residues to one or more genetic loci in the DNA.

38. The method of claim 37, wherein mapping comprises converting N4-acdC residues in the DNA into uracil residues and identifying one or more genetic loci represented by “C” in a reference genome but represented by “T” in the DNA from the subject.

39. The method of claim 37, wherein mapping comprises converting N4-acdC residues in the DNA into uracil residues and identifying one or more genetic loci represented by “C” in a reference genome but represented by “T” in the DNA from the subject.

40. The method of claim 37, wherein mapping comprises converting N4-acdC residues in the DNA into uracil residues and identifying one or more genetic loci represented by “C” in a reference genome but represented by “T” in the DNA from the subject.

41. The method of claim 37, wherein mapping comprises converting non-N4-acdC residues in the DNA into uracil residues and identifying one or more genetic loci represented by “C” in a reference genome and represented by “C” in the DNA from the subject.

42. The method of claim 37, wherein mapping comprises:
dividing the sample into first and second aliquots;
deacetylating the DNA in the first aliquot;
optionally, immunoprecipitating DNA in the two aliquots using a binding agent that specifically binds N4-acdC residue;
sequencing the DNA in the deacetylated first aliquot and in the second aliquot; and
mapping sequence reads to a reference genome, wherein genetic loci in which sequence reads are deeper for the second aliquot and then for the first acetylated aliquot represent the presence of N4-acdC.

43. The method of claim 37, wherein detecting comprises DNA sequencing, PCR, qPCR, hybridization of labeled probes against the biomarker, TaqMan amplification, or detection by molecular beacon.

44. The method of claim 37, wherein the one or more loci are biomarkers for a condition determined, for example by the method of claim 22.

45. The method of any of claims 1-46, wherein the DNA sample is obtained from a eukaryotic cell, a prokaryotic cell, an archaeal cell, a cell line, a tissue, an organ or a bodily fluid.

46. The method of claim 37, wherein the bodily fluid is blood, plasma, serum, saliva, sputum, mucus, lymphatic fluid, urine, semen, cerebrospinal fluid or amniotic fluid.

47. The method of any of claims 1-46, wherein the DNA sample contains cell-free DNA.

48. The method of any of claims 1-46, wherein the DNA fragments are obtained by sonication, shearing or enzymatic fragmentation.

49. The method of any of claims 1-46, wherein the DNA sample comprises chromatin.

50. The method of any of claims 1-46, wherein the DNA sample comprises naked DNA.

51. The method of claim 50, wherein the DNA sample comprises double-stranded DNA.

52. The method of claim 50, wherein the DNA sample comprises single-stranded DNA.

53. The method of claim 52, further comprising, either before or after step (a), denaturing the DNA.

54. The method of any of claims 1-46, wherein the N4-acdC binding agent is:
(a) an antibody,
(b) a naturally-occurring N4-acdC-binding protein, or
(c) a protein that has been engineered to bind to N4-acdC.

55. The method of claim 54, wherein enriching comprises immunoprecipitation, affinity chromatography, gel filtration or gel retardation.

56. The method of any of claims 1-46, wherein the N4-acdC binding agent comprises biotin or desthiobiotin.

57. The method of claim 54, wherein the enrichment is achieved using avidin, streptavidin or NeutrAvidin.

58. The method of any of claims 1-46, wherein one or more of the DNA fragments containing N4-acetydeoxycytine (N4-acdC) also contains a G-quadruplex.

59. The method of any of claim 1-58, comprising converting N4-acdC residues into N4-athC residues by reduction, and, as necessary, using an antibody or a protein against N4-athC rather than N4-acdC.

60. A composition comprising a DNA molecule bound to a N4-acdC binding agent.

61. The composition of claim 60, wherein the DNA molecules are purified from RNA and/or cytoplasmic proteins.

62. The composition of claim 60, wherein the N4-acdC binding agent is an antibody that specifically binds N4-acdC residues.

63. The composition of claim 62, wherein the antibody is labeled.

64. The composition of claim 63, wherein the label comprises a capture moiety (e.g., biotin) or a detectable moiety (e.g., a fluorescent molecule).

65. A composition comprising DNA molecules enriched for N4-acdC residues or N4-athC residues, wherein enrichment is at least 2×, at least 10× or at least 100× compared with a control nucleic acid from the same species as the DNA molecules.

66. A kit comprising:
(a) a first container containing a deacetylating agent; and
(b) a second container containing a binding agent that specifically binds DNA comprising N4-acdC residues.

67. A kit comprising:
(a) a first container containing a deacetylating agent; and
(b) a second container containing sodium bisulfite.

68. The kit of claim 67, further comprising:
(c) a third container containing a nucleophile

69. The kit of claim 67, further comprising:
(d) a third container containing a TET enzyme.

70. A kit comprising:
(a) a first container containing a methylase;
(b) a second container containing a TET enzyme;
(c) a third container containing sodium bisulfite.

71. The kit of claim 70, further comprising:
(d) a fourth container containing a deacetylating agent.

72. A kit comprising a first container containing a deacetylating agent, and a second container containing a restriction enzyme that does not recognize restriction sites having at least one acetylated nucleotide and, optionally, a third container containing a phosphatase enzyme.

73. A kit comprising a first container containing a deacetylating agent, a second container containing a reducing agent, a third container containing a deacetylase, a fourth container containing a molecular tag, and, optionally, a fifth container containing a binding agent that binds the tag.

74. A kit comprising a first container containing a deacetylating agent, and a second container containing a polymerase.

75. A kit comprising a first container containing a reducing agent, a second container containing an antibody or a protein that binds N4-athC.
